(19) United States
(12) Patent Application Publication
(10) Pub. No.: US 2025/0268542 A1
(43) Pub. Date: Aug. 28, 2025

(54) FAST PATIENT-SPECIFIC AUTOMATIC TUBE CURRENT MODULATION WITH IMAGE REGION-OF-INTEREST NOISE CONTROL FOR COMPUTED TOMOGRAPHY

(71) Applicant: CANON MEDICAL SYSTEMS CORPORATION, Tochigi (JP)

(72) Inventors: Yujie LU, Vernon Hills, IL (US); Ting XIA, Vernon Hills, IL (US); Jian ZHOU, Vernon Hills, IL (US); Liang CAI, Vernon Hills, IL (US)

(73) Assignee: CANON MEDICAL SYSTEMS CORPORATION, Tochigi (JP)

(21) Appl. No.: 18/586,115

(22) Filed: Feb. 23, 2024

(51) Int. Cl. A61B 6/03; A61B 6/40; A61B 6/46; G06T 7/11; G06T 11/00

(52) U.S. Cl. CPC A61B 6/032; A61B 6/405; A61B 6/469; G06T 7/11; G06T 11/006; G06T 2207/10081; G06T 2207/20104

(57) ABSTRACT

A method, apparatus, and computer-readable storage medium for controlling X-ray computed tomography (CT) imaging. A first set of projection data is acquired in a first CT scan of an object with a CT imaging apparatus. The first CT image data is reconstructed from the first set of projection data. X-ray tube current modulation information is determined for a second CT scan of the object, based on a noise propagation model between X-ray projection data and CT image data, and using, as inputs, the obtained first set of projection data, information indicating an imaging region-of-interest (ROI) for the second CT scan, and a target image quality level in the imaging ROI. The second CT scan of the object is obtained based on the obtained X-ray tube current modulation information.

### FIELD

The present disclosure relates to a method, apparatus, and non-transitory computer-readable storage medium for fast patient-specific image region-of-interest noise control in AEC prediction.

### BACKGROUND

In computed tomography (CT), a good way to reduce the patient dose with desirable diagnostic image quality is by modulating the X-ray tube current, i.e., using an automatic exposure control (AEC) method. The AEC method has been extensively used in routine clinical scanning for different protocols and anatomies of the body. However, the limited patient information available before a normal scan has an effect on the AEC prediction. Further, patient variations such as patient size, anatomical difference, and location during the scan further increase the difficulty of the AEC prediction. In addition, most of the image quality requirement is task dependent, which becomes complicated when using different protocols. Therefore, accurate AEC is still a challenging problem especially for a patient-specific scan.

Due to pre-scan limitations, current AEC methods are usually based on 2D radiographic images (typically one or two projection views). Thus, it is difficult to acquire sufficient tomographic image information. In addition, simple-model or look-up-table methods with limited two-dimensional radiographic images cannot meet the requirement of task-dependent patient-specific AEC.

Most CT vendors provide an AEC function for clinical scans. Unfortunately, due to the limitations of the simple model and pre-acquisition patient information, it is difficult to perform patient-specific, region-of-interest AEC using current methods.

Recently, organ-based tube current modulation techniques have been developed to reduce the radiation dose of sensitive organs. In one technique, a coarse CT reconstruction, an organ segmentation, and an estimation of the dose distribution can be provided in real time, for example, by applying machine-learning techniques. Using this information, the technique determines a tube current curve that minimizes a patient risk measure, for example, the effective dose, while keeping the image quality constant.

The foregoing “Background” description is for the purpose of generally presenting the context of the disclosure. Work of the inventors, to the extent it is described in this background section, as well as aspects of the description which may not otherwise qualify as prior art at the time of filing, are neither expressly nor impliedly admitted as prior art against the present disclosure.

### SUMMARY

An aspect of the present disclosure is a method of performing X-ray computed tomography (CT) imaging the method can include obtaining a first set of projection data acquired in a first CT scan of an object with a CT imaging apparatus; obtaining first CT image data reconstructed from the obtained first set of projection data; determining X-ray tube current modulation information for a second CT scan of the object, based on a noise propagation model between X-ray projection data and CT image data, and using, as inputs, the obtained first set of projection data, information indicating an imaging region-of-interest (ROI) for the second CT scan, and a set target image quality level for the imaging ROI; and performing the second CT scan of the object based on the obtained X-ray tube current modulation information.

A further aspect of the present disclosure is a non-transitory computer-readable medium storing computer-executable instructions for causing a computer to perform a method of X-ray computed tomography (CT) imaging, the method including obtaining image projection data from a pre-scan of an object using a CT apparatus; performing an analytical reconstruction of the obtained image projection data to obtain a reconstructed image; performing sparse sampling of image slices of the reconstructed image to generate a sampled image; setting a region-of-interest (ROI) in the sampled image; determining an automatic exposure control (AEC) curve using a noise propagation model, based on a set target image quality, the set ROI, and the obtained image projection data; and performing a CT scan of the object based on the determined AEC curve.

A further aspect of the present disclosure is an X-ray imaging apparatus that can include processing circuitry configured to obtain a first set of projection data acquired in a first CT scan of an object with a CT imaging apparatus; obtain first CT image data reconstructed from the obtained first set of projection data; determine X-ray tube current modulation information for a second CT scan of the object, based on a noise propagation model between X-ray projection data and CT image data, and using, as inputs, the obtained first set of projection data, information indicating an imaging region-of-interest (ROI) for the second CT scan, and a set target image quality level for the imaging ROI; and perform the second CT scan of the object based on the obtained X-ray tube current modulation information.

The foregoing general description of the illustrative embodiments and the following detailed description thereof are merely exemplary aspects of the teachings of this disclosure, and are not restrictive.

### BRIEF DESCRIPTION OF THE DRAWINGS

FIGS. 1A-1D are flowcharts of the disclosed algorithm according to exemplary embodiments described herein.

FIG. 2 illustrates an exemplary pre-scan according to an exemplary embodiment described herein.

FIG. 3 illustrates an exemplary predicted AEC curve according to an exemplary embodiment described herein.

FIG. 4 illustrates processed pre-scan images.

FIG. 5 illustrates an implementation of a radiography gantry included in a CT apparatus or scanner, according to an exemplary embodiment of the present disclosure.

FIG. 6 is a block diagram of an apparatus used for implementing the methods according to an exemplary aspect of the disclosure.

### DETAILED DESCRIPTION

The terms “a” or “an”, as used herein, are defined as one or more than one. The term “plurality”, as used herein, is defined as two or more than two. The term “another”, as used herein, is defined as at least a second or more. The terms “including” and/or “having”, as used herein, are defined as comprising (i.e., open language). Reference throughout this document to “one embodiment”, “certain embodiments”, “an embodiment”, “an implementation”, “an example” or similar terms means that a particular feature, structure, or characteristic described in connection with the embodiment is included in at least one embodiment of the present disclosure. Thus, the appearances of such phrases or in various places throughout this specification are not necessarily all referring to the same embodiment. Furthermore, the particular features, structures, or characteristics may be combined in any suitable manner in one or more embodiments without limitation.

Computed tomography (CT) scanners can have Automatic Exposure Control (AEC) systems, which aim to maintain image quality for patients of varying sizes, while keeping doses as low as reasonably practicable. Such systems are also designed to maintain image quality with the varying size and attenuation of an individual patient over their length. Some systems are based on a desired noise level in the image.

The present disclosure describes a method for increasing speed and maintaining or improving accuracy in the generation of a AEC curve in a main imaging scan (e.g., in a computed tomography (CT) scan, in a tomosynthesis scan, and in a VCT (X-ray volume CT) scan). The disclosure herein also describes an information processing apparatus including processing circuitry and/or computer instructions stored in a non-transitory computer readable storage medium for performing the above-noted methods.

Compared to simple-model or look-up-table methods with two-dimensional radiographic images, the disclosed methods can perform patient-specific task-dependent AEC prediction using pre-scan, three-dimensional patient information and a noise-propagation model.

Compared to other patient-specific radiation risk-based methods, the disclosed approach incorporates sparse-image slice sampling and analytical AEC calculation in order to achieve faster speeds. The sparse-image slice sampling uses only key slices and thus, can achieve high quality image scans while increasing the speed of determining an AEC curve. The AEC prediction uses pre-scan three-dimensional patient information that simplifies the calculation of the AEC curve. Further, the disclosed noise propagation model improves accuracy of the determined AEC curve. The faster speed and improved accuracy in generating the AEC curve can simplify the routine clinical scan and improve the diagnostic efficiency. Embodiments include a fast patient-specific image region-of-interest noise control AEC prediction framework with a 3D pre-scan.

Hereinafter, with reference to the accompanying drawings, an embodiment of an information processing method will be described in detail.

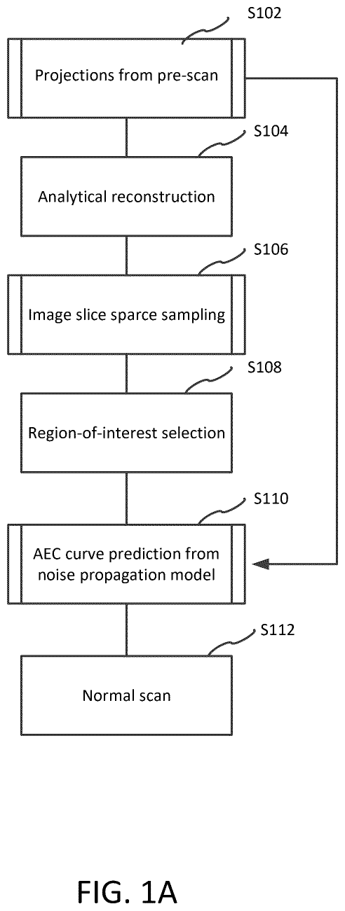

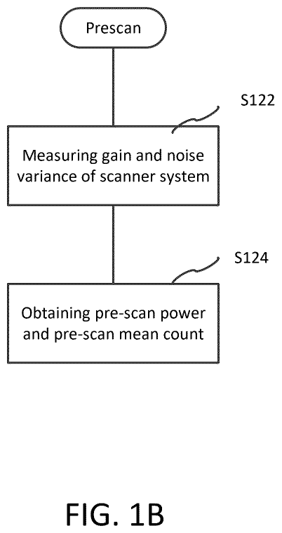

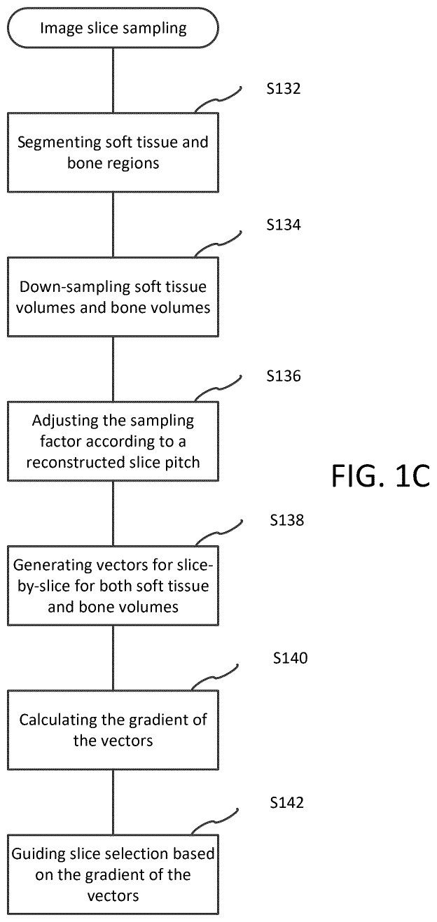

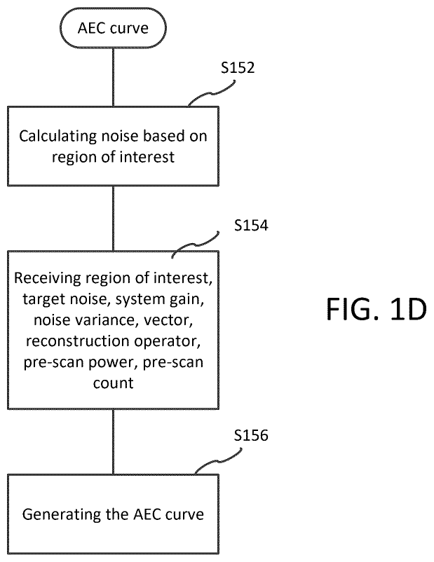

FIG. 1A is a flowchart of a patient-specific AEC method according to one embodiment of the present disclosure.

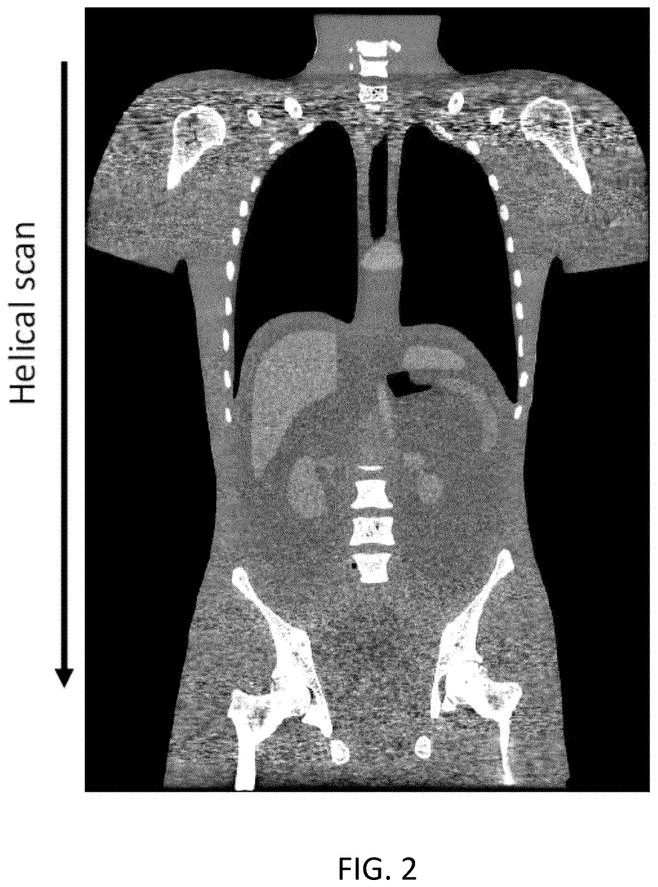

In step S102, a low-dose pre-scan is performed to acquire projection data. In one embodiment, low-dose scan can be about 50% or lower dosage than a normal scan dosage. FIG. 2 illustrates data from an exemplary pre-scan. Further details of the pre-scan process are shown in FIG. 1B, discussed below.

In step S104, fast analytical reconstruction of the projection data is performed to obtain a reconstructed image including patient anatomical information.

In step S106, slices of the reconstructed image are sampled to reduce subsequent processing time, while maintaining the AEC prediction accuracy. The sparse-sampling process is described in more detail below with respect to FIG. 1C.

In step S108, a region-of-interest (ROI) in the reconstructed image is automatically or manually selected or set.

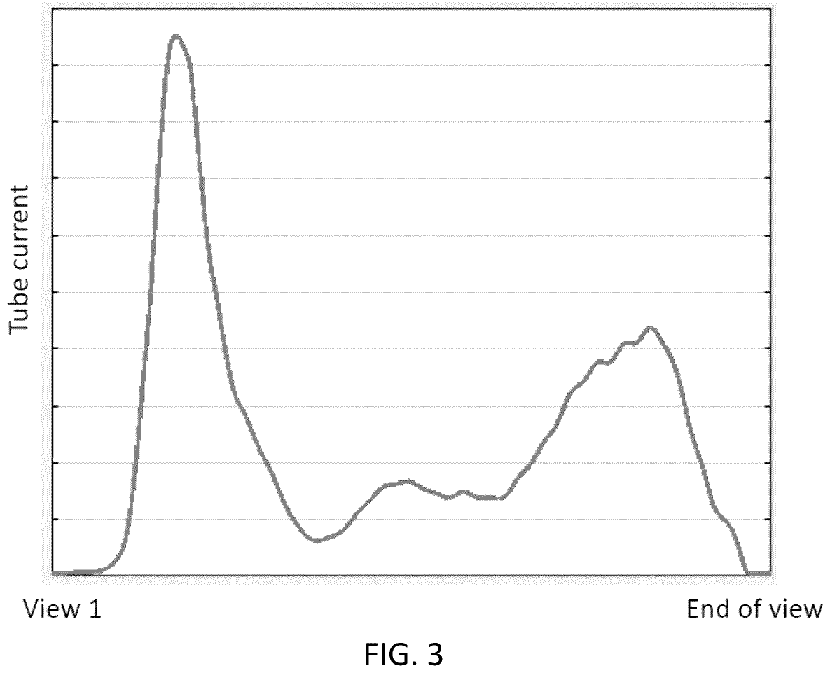

In step S110, as described in more detail below with respect to FIG. 1D, the AEC curve is determined using the disclosed noise propagation model, based on a target image quality level, such as a noise standard deviation, the set ROI, and the pre-scan projection data. FIG. 3 illustrates an exemplary determined AEC curve.

In step S112, a normal (non-pre-scan) CT scan is performed using the determined AEC curve.

As shown in FIG. 1B, in step S122, the gain and electronic noise can be measured set or measured.

In step S124, the pre-scan is performed and the pre-scan power I_{0,p} and the pre-scan mean count λₚ are obtained, as discussed in more detail below.

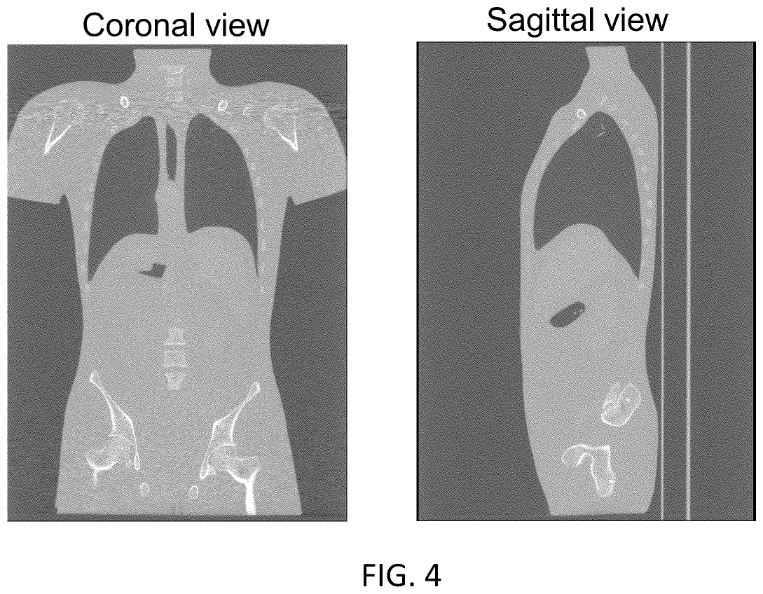

In one embodiment, the patient-specific, region-of-interest noise control AEC prediction framework can include an image sparse-sampling strategy. FIG. 1C is a flowchart of the image sparse sampling step, S106. In this process, it may not be necessary to sample every slice, but a subset of key slices may be sufficient. Although an accurate AEC curve for each slice can be obtained using a slice-by-slice strategy, it is less efficient. For example, from the processed pre-scan images (see FIG. 4) and other patient scans, the shape change of the whole body, especially in abdomen and pelvis regions, is not significant.

In S106, the image sparse-sampling strategy is applied to the three-dimensional pre-scan images to reduce the processing time. Further, before the sampling step, in step S102, the pre-scan can be performed with a low-dose helical whole-body scan.

Specifically, in FIG. 1C, in step S132, soft tissue and bone regions are segmented from a reconstructed CT volume. Image segmentation is performed to produce a segmented image B′_{seg}. Segmentation techniques include, but are not limited to, HU-based thresholding, which operates to segment out the soft-tissue region based on an HU value range. The segmentation can be used segment out bone regions. For example, at least three types of tissue can be identified: soft tissue, lung tissue, and bone. However, other types of tissue (e.g., fat) can be identified as well including other organ types, such as breast, eyes, reproductive organs, kidney, heart, liver, pancreas, and stomach. In addition, other segmentation methods can be used.

In step S134, each of the binary volumes (e.g., soft tissue and bone) can be down-sampled along the z-direction.

In step S136, the down-sampling factor can be adjusted according to the reconstructed slice pitch.

In step S138, the vectors for slice-by-slice pixel sum-up are generated for both soft tissue and bone volumes.

In step S140, the gradient of the vectors describing the change of soft tissue and bone regions is calculated.

In step S142, the sparse slice selection is guided based on the gradient of the vectors.

In this regard, normal reconstructed images have a thin image width, which has abundant information for AEC curve generation. In order to improve the efficiency, an adaptive slice sparse-sampling method was developed based on the gradient information of soft tissue and bone in the images. Specifically, the reconstructed images are segmented into the binary soft tissue and bone images using a segmentation method, such as a simple HU thresholding method. For each segmented binary volume (soft tissue and bone), each image slice is summed up to generate a vector along the z-axis direction, which can partially reflect the attenuation change slice by slice. Furthermore, the gradients of the vectors (soft tissue and bone) are obtained, which can be calculated every N slices (N can be determined based on a real case study). The gradient threshold is set to select suitable slices for further processing. All of the slices from soft tissue and bone segmentation can be combined to get the final slice list.

Referring back to step S108, in clinical applications, doctors are usually interested in some specific regions of the patient from a CT scan. The region-of-interest can be a single organ like the liver, or a region with multiple organs, such as the abdomen region. The region can be manually selected from the pre-scan images or be obtained from automatic segmentation.

In one embodiment, a patient-specific image region-of-interest noise control AEC prediction framework includes an analytical reconstruction-based noise propagation model between the projection and image region-of-interest for AEC calculation. The region-of-interest image quality, especially the signal-to-noise ratio, is important for improved diagnoses. The corresponding noise standard deviation can be used in the AEC determination process.

### Measurement Relationship Between Pre-Scan and Normal Scan

The general formula describing the detector measurements (c) and X-ray tube source is:

$$c = {I}_{0} {\int }_{e} S ( e ) exp ( - {μ}_{fit} ( e ) {l}_{fit} - {μ}_{pat} ( e ) {l}_{pat} ) de$$

where I₀ and S(e) is the power and spectrum of x-ray source and ∫ₑS(e)=1;(μ_{flt}, l_{flt}) and (μₚₐₜ, lₚₐₜ) are the attenuation and pathlength of the filter and patient, respectively. When the normal scan has the same scan geometry and protocol of the pre-scan, the mean of the measured count for each detector pixel (λ_{n,(u,v)}) can be calculated as:

$${λ}_{n , ( u , v )} = {λ}_{p ( u , v )} \frac{{I}_{0 , n}}{{I}_{0 , p}}$$

where λ_{p(u,v)} is the pre-scan mean count and I_{0,p} is the pre-scan power (see step S124 in FIG. 1B).
   Noise Model with Analytical Reconstruction

Provided B is the analytical reconstruction operator that maps the projection data γ into an image x, i.e.,

$$x = B ( y )$$

the covariance of x can be evaluated as

$$Cov ( x ) = B Cov ( y ) {B}^{′}$$

where the B is a linear operator and B′ is the transpose of B. When the region-of-interest (Ω) is selected, the noise can be calculated as:

$${s}_{Ω} = \frac{1}{\vert Ω \vert} {\sum }_{i} \sqrt{{e}_{i}^{′} B Cov ( y ) {B}^{′} {e}_{i}} .$$

### Data Noise Model

Instead of creating projection images from the pre-scan, a noise model can be constructed using Poisson noise and Gaussian noise, and propagation of noise from the projection data to the reconstructed image can be calculated. Generally, when the count level is above a predetermined threshold, the noise of the measured X-ray photon counts can be modeled with a Poisson and Gaussian distribution. In such case, the variance of the measured count (c) is

$$Var ( c ) = {a}^{2} λ + {σ}_{e}^{2}$$

where α is the gain of the data acquisition system, and σₑ² denotes the electronic noise variance (see step S122 in FIG. 1B). When the count level is below the predetermined threshold, an alternative, more accurate model can be used. In one embodiment, the noise model may be switched based on the target count level.

As the description of the model, the photon and electronic noises follow Poisson and Gaussian distributions. Regardless of the measured count level, electronic noise keeps the same distribution because it is determined by the DAS system. The photon noise can also be simulated with a Gaussian distribution when high photon counts are measured, which can further simplify the model. In the low-count level, the Poisson distribution is more accurate to describe the photon noise. Based on the measured count level, a switching method can be designed to switch the calculation method for photon noise.

CT reconstruction usually uses the logarithm of the measurement, i.e., γ. Regarding the pre- and normal-scan count relationship, variance of the normal scan data is

$$Var ( {y}_{n} ) = ( log ( \frac{{c}_{n}}{b} ) ) = \frac{{a}^{2} {λ}_{n} + {σ}_{e}^{2}}{{a}^{2} {λ}_{n}^{2}} = \frac{{a}^{2} {λ}_{p} \frac{{I}_{0 , n}}{{I}_{0 , p}} + {σ}_{e}^{2}}{{a}^{2} {{λ}_{p}^{2} ( \frac{{I}_{0 , n}}{{I}_{0 , p}} )}^{2}}$$

The normal-scan power I_{0,n} is a unknown variable, that is used to generate the AEC curve, and is view-by-view at the projection domain. The noise can thus be calculated as

$${s}_{Ω} = \frac{1}{\vert Ω \vert} {\sum }_{i} \sqrt{{e}_{i}^{′} B ( \frac{{a}^{2} {λ}_{p} \frac{{I}_{0 , n}}{{I}_{0 , p}} + {σ}_{e}^{2}}{{a}^{2} {{λ}_{p}^{2} ( \frac{{I}_{0 , n}}{{I}_{0 , p}} )}^{2}} )} {B}^{′} {e}_{i}$$

### Current Modulation

When combining the above equations for Var(γₙ) and so, the relationship of the noise standard deviation of the image region-of-interest and the X-ray tube power in the normal scan can be established. When a target noise standard deviation is set up beforehand, only I_{0,n} for generating the AEC curve is an unknown variable.

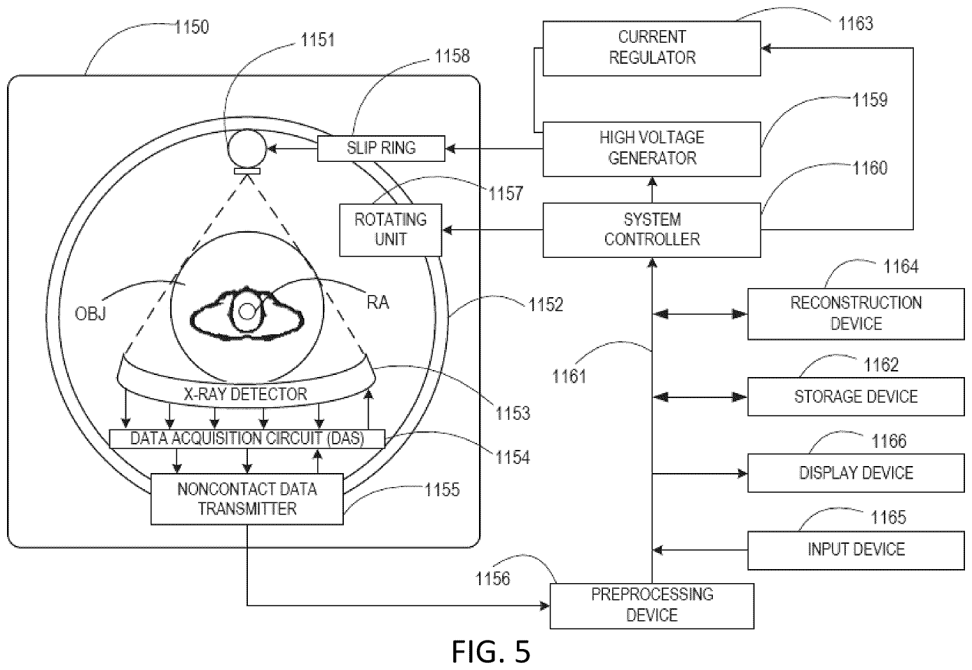

The current modulation can be performed along the z-direction or the x-y-z directions. The z-direction, or z axis, is in the direction of the bore which is “into the page” of the system (shown in FIG. 5) at locations corresponding to each of the views of the pre-scan. As used herein, the phrase “longitudinal direction” will refer to the “z” direction into the bore, in the direction of the rotation axis RA shown in FIG. 5. For the only z-direction modulation, all the projection views for each image slice can be considered consistent and I_{0,n} is calculated.

In the case of x-y-z-direction modulation, the real x-ray tube current only can have smooth view-by-view changes, which can be described by a known function (ƒ(α))·ƒ(α) can be pre-set up like sine or cosine waves. Here, a is the projection angle. I_{0,n} is then calculated to generate the normal-scan AEC. A simple example is that the AEC curve for one image slice follows the sine wave, that is sin(α_{I}_{_{o,vi}}). Here, vi is the index of the projection views for projection angle α. The new AEC curve should be I_{0,n,sin} sin(α_{I}_{_{o,vi}}). When assuming the total photons for one image slice to keep the same between z and x-y-z modulation, the following formula should be met:

$${\sum }_{v i} {I}_{0 , n , s i n} sin ( {α}_{{I}_{o , vi}} ) = {\sum }_{v i} {I}_{0 , n}$$

This formula only has one unknown variable, that is I_{0,n, sin}. Thus, it is easy to get the solution. It is noted that the method is general for more advanced ƒ(α) functions.

Referring again to FIG. 1A, in S102, I_{0,p} and λ are obtained, and a and σₑ² are scanner parameters and are measured in the scanner system, as discussed above.

In S108, in this example, the region of interest (Ω), the target noise standard deviation (S_{Ω}) is set by the doctor, for example.

In S110, the AEC curve is determined by solving the Equations for Var(γₙ) and so, where eᵢ is the i^{th} unit vector and B is analytical operator (both known). The only unknown variable is I_{0,n}, which is determined to generate the AEC curve.

According to one embodiment of the present disclosure, the above-described methods for patient-specific imaging protocols can be implemented as applied to data from a CT apparatus or scanner. FIG. 5 illustrates an implementation of a radiography gantry included in a CT apparatus or scanner. As shown in FIG. 5, a radiography gantry 1150 is illustrated from a side view and further includes an X-ray tube 1151, an annular frame 1152, and a multi-row or two-dimensional-array-type X-ray detector 1153. The X-ray tube 1151 and X-ray detector 1153 are diametrically mounted across an object OBJ on the annular frame 1152, which is rotatably supported around a rotation axis RA. A rotating unit 1157 rotates the annular frame 1152 at a high speed, such as 0.4 sec/rotation, while the object OBJ is being moved along the axis RA into or out of the illustrated page.

An embodiment of an X-ray CT apparatus according to the present disclosures will be described below with reference to the views of the accompanying drawing. Note that X-ray CT apparatuses include various types of apparatuses, e.g., a rotate/rotate-type apparatus in which an X-ray tube and X-ray detector rotate together around an object to be examined, and a stationary/rotate-type apparatus in which many detection elements are arrayed in the form of a ring or plane, and only an X-ray tube rotates around an object to be examined. The present disclosures can be applied to either type. In this case, the rotate/rotate-type, which is currently the mainstream, will be exemplified.

The multi-slice X-ray CT apparatus further includes a high voltage generator 1159 that generates a tube voltage applied to the X-ray tube 1151 through a slip ring 1158 so that the X-ray tube 1151 generates X-rays. The X-rays are emitted towards the object OBJ, whose cross-sectional area is represented by a circle. For example, the X-ray tube 1151 having an average X-ray energy during a first scan that is less than an average X-ray energy during a second scan. Thus, two or more scans can be obtained corresponding to different X-ray energies. The X-ray detector 1153 is located at an opposite side from the X-ray tube 1151 across the object OBJ for detecting the emitted X-rays that have transmitted through the object OBJ. The X-ray detector 1153 further includes individual detector elements or units and may be a photon-counting detector. In the fourth-generation geometry system, the X-ray detector 1153 may be one of a plurality of detectors arranged around the object OBJ in a 360° arrangement.

The CT apparatus further includes other devices for processing the detected signals from the X-ray detector 1153. A data acquisition circuit or a Data Acquisition System (DAS) 1154 converts a signal output from the X-ray detector 1153 for each channel into a voltage signal, amplifies he signal, and further converts the signal into a digital signal. The X-ray detector 1153 and the DAS 1154 are configured to handle a predetermined total number of projections per rotation (TPPR).

The above-described data is sent to a preprocessing device 1156, which is housed in a console outside the radiography gantry 1150 through a non-contact data transmitter 1155. The preprocessing device 1156 performs certain corrections, such as sensitivity correction, on the raw data. A memory 1162 stores the resultant data, which is also called projection data at a stage immediately before reconstruction processing. The memory 1162 is connected to a system controller 1160 through a data/control bus 1161, together with a reconstruction device 1164, input device 1165, and display 1166. The system controller 1160 controls a current regulator 1163 that limits the current to a level sufficient for driving the CT system. In an embodiment, the system controller 1160 implements optimized scan acquisition parameters.

The detectors are rotated and/or fixed with respect to the patient among various generations of the CT scanner systems. In one implementation, the above-described CT system can be an example of a combined third-generation geometry and fourth-generation geometry system. In the third-generation system, the X-ray tube 1151 and the X-ray detector 1153 are diametrically mounted on the annular frame 1152 and are rotated around the object OBJ as the annular frame 1152 is rotated about the rotation axis RA. In the fourth-generation geometry system, the detectors are fixedly placed around the patient and an X-ray tube rotates around the patient. In an alternative embodiment, the radiography gantry 1150 has multiple detectors arranged on the annular frame 1152, which is supported by a C-arm and a stand.

The memory 1162 can store the measurement value representative of the irradiance of the X-rays at the X-ray detector unit 1153. Further, the memory 1162 can store a dedicated program for executing the CT image reconstruction, material decomposition, and PQR estimation methods including methods described herein.

The reconstruction device 1164 can execute the above-referenced methods, described herein. The reconstruction device 1164 may implement reconstruction according to one or more optimized image reconstruction parameters. Further, reconstruction device 1164 can execute pre-reconstruction processing image processing such as volume rendering processing and image difference processing as needed.

The pre-reconstruction processing of the projection data performed by the preprocessing device 1156 can include correcting for detector calibrations, detector nonlinearities, and polar effects, for example.

Post-reconstruction processing performed by the reconstruction device 1164 can include generating a filter and smoothing the image, volume rendering processing, and image difference processing, as needed. The image reconstruction process may implement the optimal image reconstruction parameters derived above. The image reconstruction process can be performed using filtered back projection, iterative image reconstruction methods, or stochastic image reconstruction methods.

The reconstruction device 1164 can use the memory to store, e.g., projection data, forward projection training data, training images, uncorrected images, calibration data and parameters, and computer programs. The reconstruction device 1164 may also include processing support for machine learning, including calculating a reference data set based on the obtained spatial distribution in the soft tissue region and generating a filter by performing all or a portion of the machine learning process with the projection data set as input data and the reference data set as teacher data. Application of the machine learning, which may include application of an artificial neural network, also allows for the generation of one or more assessment values that are representative of the image quality.

The reconstruction device 1164 may be implemented by one processor individually or in a network or cloud configuration of processors. The reconstruction device 1164 can include a CPU (processing circuitry) that can be implemented as discrete logic gates, as an Application Specific Integrated Circuit (ASIC), a Field Programmable Gate Array (FPGA) or other Complex Programmable Logic Device (CPLD). An FPGA or CPLD implementation may be coded in VDHL, Verilog, or any other hardware description language and the code may be stored in an electronic memory directly within the FPGA or CPLD, or as a separate electronic memory. Further, the memory 1162 can be non-volatile, such as ROM, EPROM, EEPROM or FLASH memory. The memory 1162 can also be volatile, such as static or dynamic RAM, and a processor, such as a microcontroller or microprocessor, can be provided to manage the electronic memory as well as the interaction between the FPGA or CPLD and the memory. In an embodiment, the reconstruction device 1164 can include a CPU and a graphics processing unit (GPU) for processing and generating reconstructed images. The GPU may be a dedicated graphics card or an integrated graphics card sharing resources with the CPU, and may be one of a variety of artificial intelligence-focused types of GPUs, including NVIDIA Tesla and AMD FireStream.

Alternatively, the CPU in the reconstruction device 1164 can execute a computer program including a set of computer-readable instructions that perform the functions described herein, the program being stored in any of the above-described non-transitory electronic memories and/or a hard disc drive, CD, DVD, FLASH drive or any other known storage media. Further, the computer-readable instructions may be provided as a utility application, background daemon, or component of an operating system, or combination thereof, executing in conjunction with a processor, such as a XEON® processor from Intel® of America or an OPTERON™ processor from AMD of America and an operating system, such as Microsoft® 10, UNIX®, SOLARIS®, LINUX®, Apple MAC-OS® and other operating systems known to those skilled in the art. Further, the CPU in the reconstruction device 1164 can be implemented as multiple processors cooperatively working in parallel to perform the instructions.

In one implementation, the reconstructed images can be displayed on a display 1166. The display 1166 can be an LCD display, CRT display, plasma display, OLED, LED, or any other display known in the art.

The memory 1162 can be a hard disk drive, CD-ROM drive, DVD drive, FLASH drive, RAM, ROM, or any other electronic storage known in the art.

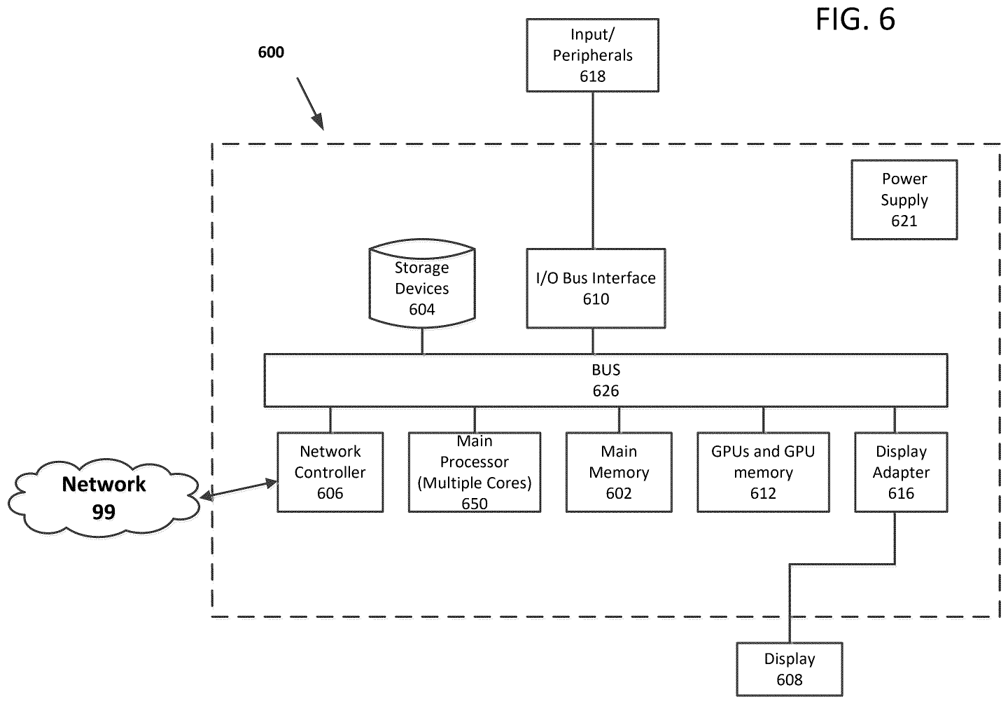

FIG. 6 is a block diagram of an apparatus used for implementing the methods according to an exemplary aspect of the disclosure. The apparatus may be a workstation running an operating system, for example Ubuntu Linux OS, Windows, a version of Unix OS, or Mac OS. The apparatus 600 may include one or more central processing units (CPU) 650 having multiple cores. The apparatus 600 may include a graphics board 612 having multiple GPUs, each GPU having GPU memory. The graphics board 612 may perform many of the mathematical operations of the disclosed machine learning methods. The apparatus 600 includes main memory 602, typically random access memory RAM, which contains the software being executed by the processing cores 650 and GPUs 612, as well as a non-volatile storage device 604 for storing data and the software programs. Several interfaces for interacting with the apparatus 600 may be provided, including an I/O Bus Interface 610, Input/Peripherals 618 such as a keyboard, touch pad, mouse, Display Adapter 616 and one or more Displays 608, and a Network Controller 606 to enable wired or wireless communication through a network 99. The interfaces, memory and processors may communicate over the system bus 626. The apparatus 600 includes a power supply 621, which may be a redundant power supply.

In some embodiments, the apparatus 600 may include a CPU and a graphics card by NVIDIA, in which the GPUs have multiple CUDA cores.

The term “processor” or “processing circuitry” used in the above description, for example, means a circuit such as a CPU, a graphics processing unit (GPU), an application specific integrated circuit (ASIC), and a programmable logic device (for example, a simple programmable logic device (SPLD), a complex programmable logic device (CPLD), and a field programmable gate array (FPGA)). When the processor is, for example, the CPU, the processor performs functions by reading and executing computer programs stored in a storage circuit. On the other hand, when the processor is, for example, the ASIC, the functions are directly incorporated in the circuit of the processor as a logic circuit instead of storing the computer programs in the storage circuit. Note that each processor of the embodiment is not limited to a case where each processor is configured as a single circuit, and one processor may be configured by combining a plurality of independent circuits to perform functions thereof. Moreover, a plurality of components in each drawing may be integrated into one processor to perform functions thereof.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

In addition to the embodiments described above, additional embodiments are described in the parentheticals set forth below.

1. A method of performing X-ray computed tomography (CT) imaging, the method comprising:
obtaining a first set of projection data acquired in a first CT scan of an object with a CT imaging apparatus, and obtaining first CT image data reconstructed from the obtained first set of projection data;
determining X-ray tube current modulation information for a second CT scan of the object, based on a noise propagation model between X-ray projection data and CT image data, and using, as inputs, at least a part of the obtained first set of projection data, information indicating an imaging region-of-interest (ROI) for the second CT scan, and a target image quality level in the imaging ROI; and
performing the second CT scan of the object based on the obtained X-ray tube current modulation information.

2. The method of claim 1, further comprising setting the imaging ROI of the second CT scan using the first CT image data.

3. The method of claim 1, wherein in the step of determining the X-ray tube current modulation information further comprising inputting at least one parameter indicating characteristics of data acquisition by the CT apparatus into the noise propagation model.

4. The method of claim 1, further comprising performing sparse sampling of the obtained first CT image data to generate second CT image data, and setting the imaging ROI in the second CT image data.

5. The method of claim 1, further comprising setting the imaging ROI based on anatomical detection processing.

6. The method of claim 1, wherein the determined tube current modulation information includes a tube current as a function of projection angle.

7. The method of claim 1, further comprising setting the imaging ROI based on an input from a user.

8. The method of claim 1, further comprising determining a size of the imaging ROI, wherein the determining step further comprises determining the X-ray tube modulation information based on the determined size of the imaging ROI.

9. A non-transitory computer-readable medium storing computer-executable instructions for causing a computer to perform a method of X-ray computed tomography (CT) imaging, the method comprising:
obtaining image projection data from a pre-scan of an object;
performing an analytical reconstruction of the obtained image projection data to obtain a reconstructed image;
performing sparse sampling of image slices of the reconstructed image to generate a sampled image;
selecting a region-of-interest (ROI) in the sampled image;
determining an automatic exposure control (AEC) curve using a noise propagation model, based on a target image quality, the selected ROI, and the image projection data; and
performing a CT scan based on the determined AEC curve.

10. The non-transitory computer-readable medium of claim 9, wherein the step of performing the sparse sampling further comprises:
segmenting soft tissue and bone regions in the reconstructed image;
generating vectors for slice-by-slice pixel summation for both the soft tissue and the bone regions;
calculating a gradient of the generated vectors; and
guiding slice selection based on the calculated gradient of the vectors.

11. The non-transitory computer-readable medium of claim 9, wherein the steps of determining the AEC curve further comprises:
obtaining a gain of the CT system, a noise variance, a pre-scan power, and a pre-scan count as pre-scan scanner-based values; and
and determining the AEC curve based on the scanner-based values.

12. The non-transitory computer-readable medium of claim 9, wherein the step of determining AEC curve further comprises determining the AEC curve based on a pre-set target noise standard deviation.

13. The non-transitory computer-readable medium of claim 10, wherein the step of determining the AEC curve further comprises determining the AEC curve based on the generated vectors.

14. The non-transitory computer-readable medium of claim 9, further comprising performing current modulation of the CT scan in a direction, wherein the direction is an axial direction of a CT system.

15. The non-transitory computer-readable medium of claim 9, further comprising performing current modulation of the CT scan based on a projection angle.

16. An X-ray imaging apparatus, comprising:
processing circuitry configured to
obtain a first set of projection data acquired in a first CT scan of an object with a CT imaging apparatus, and obtain first CT image data reconstructed from the obtained first set of projection data;
determine X-ray tube current modulation information for a second CT scan of the object, based on a noise propagation model between X-ray projection data and CT image data, and using, as inputs, at least a part of the obtained first set of projection data, information indicating an imaging region-of-interest (ROI) for the second CT scan, and a target image quality level in the imaging ROI; and
perform the second CT scan of the object based on the obtained X-ray tube current modulation information.

17. The apparatus of claim 16, wherein the processing circuitry is further configured to set the imaging ROI of the second CT scan using the first CT image data.

18. The apparatus of claim 16, wherein in determining the X-ray tube current modulation information, the processing circuitry is further configured to input at least one parameter indicating characteristics of data acquisition by the CT apparatus into the noise propagation model.

19. The apparatus of claim 16, wherein the processing circuitry is further configured to set the imaging ROI based on anatomical detection processing.

20. The apparatus of claim 16, wherein the processing circuitry is further configured to set the imaging ROI based on an input from a user.
